(19) United States
(12) Patent Application Publication
(10) Pub. No.: US 2008/0085892 A1
(43) Pub. Date: Apr. 10, 2008

(54) Liquid dosage form of acetaminophen

(76) Inventors: Sangeetha Kandeepan, Hyderabad (IN); Krishna Murthy Bhavanasi, Hyderabad (IN); Venkata Ram Mohan Rao Visinigiri, Hyderabad (IN); Nagaprasad Vishnubhotla, Hyderabaad (IN); Sivakumaran Meenakshisunderam, Hyderabad (IN)

Correspondence Address: Jay R Akhave, # 612, 2058 N Mills Ave, Claremont, CA, 91711, US

(21) Appl. No.: 11/897,765

(22) Filed: Aug. 31, 2007

(30) Foreign Application Priority Data
Aug. 31, 2006 (IN) 1573/CHE/2006

(51) Int. Cl. A61K 31/5415; A61K 31/075; A61K 31/135; A61K 31/164; A61K 31/192; A61P 43/00; A61K 31/4355; A61K 31/44; A61K 31/4427

(52) U.S. Cl. 514/226.5; 514/282; 514/289; 514/343; 514/357; 514/568; 514/625; 514/653; 514/718

(57) ABSTRACT

The present invention relates to a liquid dosage form comprising pharmaceutically insoluble and unpleasant active drug and liquid excipient base. In particular, the invention relates to a liquid dosage form comprising Acetaminophen and liquid excipient base as a solubilizer.

### CROSS REFERENCE TO THE RELATED APPLICATION

This application claims priority from the Indian Application No. 1573/CHE/2006 with a filing date of Aug. 31, 2006.

### FIELD OF THE INVENTION

The present invention relates to a liquid dosage form comprising insoluble and unpleasant active drug and a liquid excipient base. In particular, the invention relates to a liquid dosage form comprising acetaminophen and a liquid excipient base as a solubilizer.

The present invention also relates to a process for the preparation of liquid dosage form comprising acetaminophen and a liquid excipient base as a solubilizer.

### BACKGROUND OF THE INVENTION

Drugs can be administered orally by means of various dosage form i.e. solid dosage forms like tablet, capsule or caplet and or liquid forms such as solution, syrup, elixir and suspension etc. The liquid dosage forms are preferred over solid dosage forms mainly for children, older person and many other persons including disabled or incapacitated patients as they often have trouble in taking solid dosage form. A common problem associated with liquid pharmaceutical dosage forms is poor aqueous solubility and disagreeable taste that a pharmaceutically active agent may often manifest when administered in a liquid dosage form.

Acetaminophen is chemically known as N-(4-hydroxyphenyl)acetamide. Acetaminophen is slightly soluble in water and has bitter taste. It is a non-opiate, non-salicylate analgesic and antipyretic drug.

Guaifenesin is chemically known as 3-(2-methoxyphenoxy)-1,2-propanediol. Guaifenesin is slightly soluble (i.e., about 5% wt.) in water and has bitter taste. It is an expectorant usually taken orally in acute respiratory tract infections and also an ingredient in many over-the-counter cough and cold products.

To overcome the problems associated with slightly soluble and unpleasant active drugs, various prior art patents disclose the use of liquid excipient bases as solubility enhancer and taste masking effect. Given below there are some patents and publications, which disclose liquid dosage forms that, comprises of slightly soluble and bitter tasting drugs.

U.S. Pat. No. 5,154,926 describes a syrup composition comprising acetaminophen or phenobarbital, a polyhydric alcohol and/or a polymer of a polyhydric alcohol, and a water soluble macromolecule maintaining weight ratio of phenobarbital to polyhydric alcohol and/or the polymer of the polyhydric alcohol mainly comprises of polyethylene glycol having molecular weight of 300-400 from 1:20 to 1:100, and the weight ratio of phenobarbital to the water soluble macromolecule is from 1:1 to 1:20. Similarly the ratio between the acetaminophen to polyhydric alcohol is maintained 1:1 to 1:10 and the weight ratio of acetaminophen to the water soluble macromolecule is from 1:0.1 to 1:2.

U.S. Pat. No. 5,484,606 discloses a process for reducing precipitation of difficultly soluble pharmaceutical active, which basically consists of dextromethorphan HBr, pseudoephedrine HCl and acetaminophen in a mixture of polyethylene glycol, polyvinylpyrrolidone and propylene glycol.

U.S. Pat. No. 5,510,389 discloses a concentrated acetaminophen solution which basically comprises of acetaminophen alone or in combination with dextromethorphan HBr, Beckman Rearrangement catalyst, polyethylene glycol having molecular weight less than 1000, propylene glycol and polyvinyl pyrrolidone.

U.S. Pat. Nos. 5,563,177 and 5,616,621 discloses a liquid pharmaceutical composition which comprises liquid excipient base mainly comprises of polyethylene glycol of molecular weight 1000 to 2000 maintaining the weight in the ratio of 100:1 to 20:1 with sodium carboxy methylcellulose for administration of relatively large amounts of unpleasant tasting medicines including antihistamines, decongestants, antitussives, expectorants, non-steroidal anti-inflammatory drugs (NSAIDs) and other analgesic drugs such as acetominophen and phenacetin.

U.S. Pat. No. 5,763,449 describes a pharmaceutical composition of bitter tasting and slightly soluble drug selected from the group consisting of acetaminophen, terfenadine, guaifenesin, pseudoephedrine hydrochloride, etc dissolved or dispersed in an aqueous medium that is free of ethanol. The aqueous medium mainly comprises of water, about 5 to 10 weight percent of polyvinylpyrrolidone, about 45 to 55 weight percent of C₃ to C₆ polyol, 0.01 to 0.5 weight percent of ammonium glycyrrhizinate and one or more flavorants.

Similarly, U.S. Pat. No. 6,391,886 describes an oral composition containing oral mucosal tissue irritating drugs selected from the group consisting of dextromethorphan, acetaminophen, pseudoephedrine, guaifenesin, ambroxyl, etc and their mixtures, which was mitigated by the use of variety of coolants and sweeteners selected from the group consisting of sodium saccharine, potassium acesulfame, sucralose, aspartame, monoammonium glycyrrhizinate and neohesperidin dihydrochalcone.

U.S. Pat. No. 7,101,572 describes liquid composition containing an effective amount of an unpleasant tasting drug dissolved or dispersed in an aqueous excipient base, which comprises polyvinyl pyrrolidone and/or copolyvidone, and high molecular weight polyethylene glycol of about 2000 to 8000.

US 2006/0013834 discloses a liquid composition that comprises a pharmaceutically effective amount of a drug dissolved or dispersed in an aqueous medium. The aqueous medium consists essentially of water, about 3% to about 10% w/v polyvinylpyrrolidone, about 60% to about 75% w/v of C₃-C₆ polyol that includes more than 55% w/v of a non-reducing disaccharide, trisaccharide or tetrasaccharide such as sucrose, optionally about 0.01% to about 0.5% w/v of a glycyrrhetic acid, glycyrrhizinate derivative or salt thereof, and one or more flavorants.

Pharmaceutically acceptable liquid excipient bases as solubilizer for administration of slightly soluble and unpleasant tasting drugs are well known in the art. Even though, many liquid excipient bases are well known in the prior art, still slightly soluble and unpleasant tasting medicines alone or in combination pose challenges to one skilled in the art to provide better liquid dosage form.

In continuing efforts to develop oral liquid dosage forms, the inventors of the present invention found that a liquid excipient base containing high concentrations of polyethylene glycol of molecular weight less than 1000 and propylene glycol, enhances the solubility and also masks the taste of unpleasant drugs in an acceptable manner.

### OBJECTIVE OF THE PRESENT INVENTION

Accordingly, the main objective of the present invention is to provide an oral liquid dosage form comprising slightly soluble and bitter tasting acetaminophen.

### SUMMARY OF THE INVENTION

The present invention provides an oral liquid dosage form comprising acetaminophen, one or more pharmaceutically active compounds and liquid excipient base as a solubilizer, wherein the liquid excipient base comprises a mixture of polyethylene glycol having molecular weight less than 1000 and propylene glycol.

### DETAILED DESCRIPTION OF THE INVENTION

In an embodiment of the present invention, the polyethylene glycol of the present invention has molecular weight less than 1000, more preferably polyethylene glycol having molecular weight between 200 to 800 is used. Polyethylene glycols having molecular weight less than 1000 enhance the rate of release of drug. In addition to that, they also mask the disagreeable taste of the drug and have low toxicity in comparison to high molecular weight polyethylene glycol.

In another embodiment, the oral liquid dosage further comprises one or more pharmaceutically active compounds selected from antihistamines such as chlorpheniramine, brompheniramine, triprolidine and the like, decongestants such as pseudoephedrine, phenylpropolamine and the like, antitussives such as caramiphen, dextromethorphan and codeine and the like, expectorants such as guaifenesin, terpin hydrate and potassium guaicolsulfonate and the like, non-steroidal anti-inflammatory drugs (NSAIDs) such as ibuprofen, naproxen, piroxicam, indomethacin and the like and analgesics such as phenacetin.

In yet another embodiment of the present invention, the amount of acetaminophen used may be in the range of about 20-40 mg/ml.

In yet another embodiment of the present invention, the amount of another pharmaceutically active compound, preferably guaifenesin or dextromethorphan HBr used may be in the range of 5-30 mg/ml.

Yet in another embodiment, the liquid excipient base further comprises of water.

In yet another embodiment, the oral liquid dosage form of the present invention further comprise one or more pharmaceutically acceptable excipients such as viscosity modifier, preservatives, sweetening agent, coloring agents and flavouring agents.

Suitable viscosity modifier useful according to the present invention includes cellulosic materials, xanthum gum and gum arabic and the like or mixture thereof. The cellulosic material are selected from methylcellulose, hydroxyethyl cellulose, hydroxypropylcellulose, hydroxyethyl methylcellulose, hydroxypropyl methylcellulose, carboxy methylcellulose and its salt and the like or mixture thereof.

In another embodiment, the weight ratio of polyethylene glycol to viscosity modifier is maintained in the ratio of 120:1 to 200:1, more preferably, in the range of 120:1 to 180:1 to maintain the optimum viscosity of the composition.

Suitable preservatives useful according to the present invention include sodium benzoate, citric acid and the like. The preservative may be used in the range of about 0.1 to about 0.5 gram per 100 mL.

Suitable sweetening agents useful according to the present invention include mainly sucrose, sorbitol, sucralose and the like. The sweetening agents may be used in the range of 0.1 to 25 gram per 100 mL

Suitable flavouring agents useful according to the present invention include natural peppermint, menthol, thymol and the like, which may be present in permissible amount to provide additional taste masking effect.

Colouring agents may also be incorporated to provide an appealing colour to the taste masked liquid dosage form, preferably comprises of FD & C blue in permissible amount.

In yet another embodiment, the pH of the liquid excipient base is maintained in the range of 4.5 to 6.5.

In yet another embodiment of the present invention, the viscosity of the liquid excipient base is maintained in the range: of 80-140 CPs measured with Brookfield viscometer Spindle No: 1(LV1) at 40 RPM and temperature 25±3° C.

In another embodiment of the present invention, there is provided a manufacturing process for an oral liquid dosage form comprising acetaminophen, one or more pharmaceutically active compounds and liquid excipient base as a solubilizer, wherein the liquid excipient base comprises a mixture of polyethylene glycol having molecular weight less than 1000 and propylene glycol, which comprises the steps of

i). preparing a liquid excipient base comprising polyethylene glycol having molecular weight less than 1000 and propylene glycol,

ii). dissolving acetaminophen in the liquid excipient base by stirring for a period of 15 to 60 minutes,

iii). adding guaifenesin to the solution obtained in step (ii) by stirring,

iv). preparing a solution of preservatives and sweetening agent in water separately,

v). adding the solution of step (iv) to the solution of step (iii) by stirring,

vi). adding a solution containing viscosity modifier to the solution obtained in step (v),

vii). adding flavouring agent and colouring agent with the solution obtained in step (vi) and

viii). finally make up the volume with purified water and filtering the solution through filters.

The following examples further exemplify the inventions and are not intended to limit the scope of the inventions. It is obvious to those skilled in the art to find out the composition for other dosage forms and substitute the equivalent excipients as described in this specification or with the one known to the industry.

### EXAMPLE 1

| | | Qty in | |
|---|---|---|---|
| S. No | Ingredients | mg/ml | |
| 1 | Acetaminophen | 33.33 | mg |
| 2 | Guaifenesin | 13.33 | mg |
| 3 | Propylene glycol | 125.00 | mg |
| 4 | Polyethylene glycol 600 | 350.00 | mg |
| 5 | Sucrose 60/200 | 250.00 | mg |
| 6 | Sorbitol 70/70 | 125.00 | mg |
| 7 | Sucralose | 5.00 | mg |
| 8 | Citric acid | 0.70 | mg |
| 9 | Sodium benzoate | 2.00 | mg |
| 10 | Sodium carboxy methylcellulose | 2.00 | mg |
| 11 | Natural peppermint flavor #104 | 2.00 | mg |
| 12 | FD & C Blue # 1 | 0.01 | mg |

| | | |
|---|---|---|
| 13 | Purified water | qs |

1. a solution of liquid excipient was prepared which comprises of polyethylene glycol 600 and propylene glycol,
2. Acetaminophen was dissolved in a solution of liquid excipient base under stirring for about 15 to 60 minutes,
3. Guaifenesin was added to the acetaminophen solution obtained in step (2) under stirring,
4. sodium benzoate, sucralose, citric acid, sorbitol was added successively to 20% of purified water in a flask and stirred,
5. the solution of step (4) was added to acetaminophen solution obtained in step (3) under stirring,
6. to the solution of step (5) sodium carboxy methylcellulose soaked in 5% of purified water was added and stirred for 10 min,
7. peppermint flavor, FD & C blue #1 was added to the solution of steps (6) and
8. finally the volume was made with purified water and filtered through 5μfilters

### EXAMPLE 2

| | | Qty in | |
|---|---|---|---|
| S. No | Ingredients | mg/ml | |
| 1 | Acetaminophen | 33.33 | mg |
| 2 | Guaifenesin | 13.33 | mg |
| 3 | Propylene Glycol | 125.00 | mg |
| 4 | Polyethylene Glycol 600 | 300.00 | mg |
| 5 | Sucrose 60/200 | 200.00 | mg |
| 6 | Sorbitol 70/70 | 125.00 | mg |
| 7 | Sucralose | 5.00 | mg |
| 8 | Citric Acid | 0.70 | mg |
| 9 | Sodium Benzoate | 2.00 | mg |
| 10 | Sodium CMC | 2.00 | mg |
| 11 | Natural Peppermint Flavor #104 | 2.00 | mg |
| 12 | FD & C Blue # 1 | 0.01 | mg |

| | | |
|---|---|---|
| 13 | Purified Water | qs |

The oral liquid dosage form disclosed in example 2 was prepared by the similar procedure described in example 1.

We claim:

1. A oral liquid dosage form comprising acetaminophen, one or more pharmaceutically active compounds and a liquid excipient base as a solubilizer, wherein the liquid excipient base comprises a mixture of polyethylene glycol having molecular weight less than 1,000 and propylene glycol.

2. The liquid dosage form as claimed in claim 1, wherein one or more pharmaceutically active compounds are selected from antihistamines such as chlorpheniramine, brompheniramine, triprolidine; decongestants such as pseudoephedrine, phenylpropanolamine; antitussives such as caramiphen, dextromethorphan and codeine; expectorants such as guaifenesin, terpin hydrate and potassium guaicolsulfonate; non-steroidal anti-inflammatory drugs (NSAIDs) such as ibuprofen, naproxen, piroxicam, indomethacin and analgesics such as phenacetin.

3. The liquid dosage form as claimed in claim 1, further comprise one or more pharmaceutically acceptable such as viscosity modifier, preservatives, sweetening agent, coloring agents and flavouring agents.

4. The liquid dosage form as claimed in claim 3, wherein the viscosity modifier is selected from cellulosic materials, xanthum gum and gum arabic or a mixture thereof.

5. The liquid dosage form as claimed in claim 3, wherein the preservative is selected from sodium benzoate, citric acid, propylene glycol or a mixture thereof.

6. The liquid dosage form as claimed in claim 3, wherein the sweetening agent is selected from sucrose, sorbitol, sucralose or a mixture thereof.

7. The liquid dosage form as claimed in claim 3, wherein the flavouring agent is selected from natural peppermint, menthol, thymol or a mixture thereof.

8. The liquid dosage form as claimed in claim 1, wherein the amount of acetaminophen used is in the range of about 20-40 mg/ml.

9. The liquid dosage form as claimed in claim 1, wherein the pH of the liquid excipient base is in the range of 4.5 to 6.5.

10. A process for manufacturing oral liquid dosage form comprising acetaminophen, one or more pharmaceutically active compounds and liquid excipient base as a solubilizer, wherein the liquid excipient base comprises a mixture of polyethylene glycol having molecular weight less than 1000 and propylene glycol, that comprises the steps of:
i). preparing a liquid excipient base comprising polyethylene glycol having molecular weight less than 1000 and propylene glycol,
ii). dissolving acetaminophen in the liquid excipient base by stirring for a period of 15 to 60 minutes,
iii). adding guaifenesin to the solution obtained in step (ii) by stirring,
iv). preparing a solution of preservatives and sweetening agent in water separately,
v). adding the solution of step (iv) to the solution of step (iii) by stirring,
vi). adding a solution containing viscosity modifier to the solution obtained in step (v),
vii). adding flavouring agent and colouring agent with the solution obtained in step (vi) and
viii). finally make up the volume with purified water and filtering the solution through filters.
